(19) United States
(12) Patent Application Publication
(10) Pub. No.: US 2026/0249032 A1
(43) Pub. Date: Aug. 27, 2026

(54) TUBE SUPPORT APPARATUS AND METHODS

(71) Applicant: WARRIORNP LLC, Bloomfield Hills, MI (US)

(72) Inventors: Jessica Harnish, Bloomfield Hills, MI (US); Brian Smith, Auburn Hills, MI (US); Steven Fileccia, Auburn Hills, MI (US)

(21) Appl. No.: 19/489,647

(22) PCT Filed: Jun. 6, 2024
(86) PCT No.: PCT/US2024/032808

§ 371 Date: Dec. 3, 2025

Related U.S. Application Data
(60) Provisional application No. 63/471,346, filed on Jun. 6, 2023.
(60) Provisional application No. 63/544,844, filed on Oct. 19, 2023.

(51) Int. Cl. A61M 16/04; A61M 25/02

(52) U.S. Cl. CPC A61M 16/0497; A61M 2025/022; A61M 2209/088

(57) ABSTRACT

A method of securing one or more tubes inserted within a patient may include removing an end portion of a tube support apparatus from a channel in a cheek pad, and securing the one or more tubes by inserting a leg portion of a clip into the channel and placing the one or more tubes within one or more grooves of the clip.

### CROSS-REFERENCED TO RELATED APPLICATION

This application claims priority to U.S. Provisional Application No. 63/471,346, which was filed on Jun. 6, 2023, and U.S. Provisional Application No. 63/544,844, which was filed on Oct. 19, 2023.

### BACKGROUND

Endotracheal intubation is a medical procedure in which a flexible tube is placed through the mouth or nose into the windpipe (trachea) to help a patient breathe. In some instances, endotracheal intubation is an emergency procedure that is performed on a patient who may be unconscious or who cannot breathe on their own.

Nasogastric tubes or orogastric tubes are small tubes placed either through the nose or the mouth and end with the tip in the stomach or the small intestines. Nasogastric tubes or orogastric tubes may be used for feedings, medication administration, or removal of contents from the stomach via aspiration, suction, or gravity drainage.

Gastric suction is a procedure to empty the contents of your stomach. Gastric suction is performed to empty the contents of the stomach before it passes through the rest of the digestive tract.

### SUMMARY

In some aspects, the techniques described herein relate to a method of securing one or more tubes inserted within a patient, the method including: removing an end portion of a tube support apparatus from a channel in a cheek pad; and securing the one or more tubes by inserting a leg portion of a clip into the channel and placing the one or more tubes within one or more grooves of the clip.

In some aspects, the techniques described herein relate to a method, wherein the leg portion includes one or more protrusions.

In some aspects, the techniques described herein relate to a method, wherein the one or more protrusions include one or more ribs.

In some aspects, the techniques described herein relate to a method, wherein the clip includes as second leg providing the one or more grooves.

In some aspects, the techniques described herein relate to a method, wherein the securing step includes securing the one or more tubes between the second leg and the cheek pad.

In some aspects, the techniques described herein relate to a method, wherein the one or more tubes include one or more of a nasal cannula, a gastric suction tube and a feeding tube.

In some aspects, the techniques described herein relate to a method, wherein the one or more grooves includes a first groove dimensioned to receive a tube of a first diameter, and a second groove dimensioned to receive a tube of a second, different diameter.

In some aspects, the techniques described herein relate to a method, wherein the cheek pad includes a main body portion and a raised portion, and the channel is provided in the raised portion.

In some aspects, the techniques described herein relate to a tube support apparatus, including: a brace for attachment to a patient's face and including an end portion; a support extending from the brace and including a groove for receiving a tube; a pad for attachment to the patients face including a channel for receiving the end portion, wherein the end portion includes a protrusion configured to engage an edge at a lateral end of the channel to inhibit the end portion from being pulled out of the channel.

In some aspects, the techniques described herein relate to an apparatus, wherein the pad includes a main body portion and a raised portion, and the channel is provided in the raised portion.

In some aspects, the techniques described herein relate to an apparatus, wherein the edge extends from the lateral end of the channel to the main body portion.

In some aspects, the techniques described herein relate to an apparatus, wherein the protrusion is shaped like a hook.

In some aspects, the techniques described herein relate to an apparatus, including a shelf within the groove for being received against the tube.

In some aspects, the techniques described herein relate to an apparatus, including a clamp for securing the tube within the groove.

In some aspects, the techniques described herein relate to an apparatus, wherein the clamp includes a plastic body.

In some aspects, the techniques described herein relate to a method of securing a tube within a patient, the method including: securing the tube to a tube support apparatus including an end portion; adhering a pad to a cheek of the patient; and passing the end portion through a channel in the pad, wherein the end portion includes a protrusion configured to engage an edge at a lateral end of the channel to inhibit the end portion from being pulled out of the channel.

In some aspects, the techniques described herein relate to a method, wherein the tube support apparatus includes a brace for attachment to the patient's face and a support extending from the brace and including a groove for receiving the tube.

In some aspects, the techniques described herein relate to a method, wherein the tube support apparatus includes a second end portion, the method including: adhering a second pad to a second cheek of the patient, and passing the second end portion through a second channel in the second pad, wherein the second end portion includes a second protrusion configured to engage a second edge at a lateral end of the second channel to inhibit the second end portion from being pulled out of the second channel.

In some aspects, the techniques described herein relate to a method, wherein the protrusion is shaped like a hook.

In some aspects, the techniques described herein relate to a method, wherein the securing step includes clamping the tube within a groove in the tube support apparatus.

In some aspects, the techniques described herein relate to a tube support apparatus, including: a brace for attachment to a patient's face; a support extending from the brace and including a groove for receiving a tube; a first protrusion within the groove for being received against the tube; a clamp for surrounding the support and the tube; and a second protrusion extending from the clamp for being received against the tube.

In some aspects, the techniques described herein relate to an apparatus, wherein the clamp includes a plastic body.

In some aspects, the techniques described herein relate to an apparatus, wherein the clamp is generally C-shaped.

In some aspects, the techniques described herein relate to an apparatus, wherein the clamp includes an open circumferential portion.

In some aspects, the techniques described herein relate to an apparatus, wherein the support includes a second groove configured to receive a second tube.

In some aspects, the techniques described herein relate to an apparatus, wherein the second tube is a feeding tube or a gastric suction tube.

In some aspects, the techniques described herein relate to an apparatus, wherein the support includes a third groove, and the first, second, and third grooves are sized differently, and the third groove is closer circumferentially to the second groove than the first groove.

In some aspects, the techniques described herein relate to an apparatus, wherein the open circumferential portion is sized such that it is received over a radially outer surface of the support when tubes are to be secured in the first and second groove.

In some aspects, the techniques described herein relate to an apparatus, wherein the open circumferential portion has an arc length greater than or equal to the arc length of the largest groove, and the open circumferential portion arc length is less than the arc length of at least one of the radially outer surfaces of the support.

In some aspects, the techniques described herein relate to an apparatus, wherein the first and second groove are positioned such that when the endotracheal tube is to be adjusted or replaced, the clamp can be rotated in a clockwise or counterclockwise direction so that the open circumferential portion is circumferentially aligned with the first groove, while the clamp secures the second tube in the second groove.

In some aspects, the techniques described herein relate to an apparatus, wherein the first and second groove are positioned such that when the second tube is to be adjusted or replaced, the clamp can be rotated in a clockwise or counterclockwise direction so that the open circumferential portion is circumferentially aligned with the second groove, while the clamp secures the endotracheal tube in the first groove.

In some aspects, the techniques described herein relate to an apparatus, wherein the support includes a slot for receiving the second protrusion.

In some aspects, the techniques described herein relate to an apparatus, wherein the first protrusion is a shelf.

In some aspects, the techniques described herein relate to an apparatus, wherein the second protrusion is a shelf.

In some aspects, the techniques described herein relate to a method including: removing an endotracheal tube holder from a cheek pad; inserting one or more tubes within a patient; securing the one or more tubes to the cheek pad using an M-shaped clip.

In some aspects, the techniques described herein relate to a method, wherein the M-shaped clip includes a central leg, and the securing step includes inserting the central leg into a channel of the cheek pad.

In some aspects, the techniques described herein relate to a method, wherein the central leg includes one or more protrusions.

In some aspects, the techniques described herein relate to a method, wherein the one or more protrusions include one or more ribs.

In some aspects, the techniques described herein relate to a method, wherein the M-shaped clip includes an outer leg having a groove, and the securing step includes placing the outer leg over one of the one or more tubes such that the one of the one or more tubes is received in the groove.

In some aspects, the techniques described herein relate to a method, wherein the one or more tubes include a nasal cannula and a feeding tube.

In some aspects, the techniques described herein relate to a tube support apparatus, including: a brace for attachment to a patient's face and including an end portion; a support extending from the brace and including a groove for receiving a tube; a pad for attachment to the patients face including a channel for receiving the end portion, wherein the end portion includes a protrusion configured to engage a raised edge at a lateral end of the channel to inhibit the end portion from being pulled out of the channel.

These and other features may be best understood from the following specification and drawings, the following of which is a brief description.

### BRIEF DESCRIPTION OF THE DRAWINGS

FIG. 1 illustrates an example tube support apparatus.

FIG. 2 illustrates another example tube support apparatus.

FIG. 3 illustrates the support and clamp of the example tube support apparatus of FIG. 2.

FIG. 4 illustrates the support and clamp of the example tube support apparatus of FIGS. 2 and 3.

FIG. 5 illustrates a top view of the support and clamp of the example tube support apparatus of FIGS. 2-4.

FIG. 6 illustrates a top view of the support and clamp of the example tube support apparatus of FIGS. 2-5 with tubes secured in two of the grooves.

FIG. 7 illustrates a first disengaged position of the clamp and support of the example tube support apparatus of FIGS. 2-6.

FIG. 8 illustrates a second disengaged position of the clamp and support of the example tube support apparatus of FIGS. 2-7.

FIG. 9 illustrates a third disengaged position of the clamp and support of the example tube support apparatus of FIGS. 2-8.

FIG. 10 illustrates another example tube support apparatus.

FIG. 11 illustrates the example tube support apparatus of FIG. 10.

FIG. 12 illustrates the example tube support apparatus of FIGS. 10-11.

FIG. 13 illustrates the support and clamp of the example tube support apparatus of FIGS. 10-12.

FIG. 14 illustrates the support and clamp of the example tube support apparatus of FIGS. 10-13.

FIG. 15 illustrates the example tube support apparatus of FIGS. 10-14 utilized on a patient after an endotracheal intubation.

FIG. 16A illustrates the example tube support apparatus of FIGS. 10-15 utilized on a patient after an endotracheal intubation and further supporting a nasogastric tube.

FIG. 16B illustrates the example tube support apparatus of FIGS. 10-15 utilized on a patient after an endotracheal intubation and further supporting an orogastric tube and in a flipped orientation to that shown in FIG. 16A.

FIG. 17 the example tube support apparatus of FIGS. 10-16B utilized on a patient after an endotracheal intubation.

FIG. 18 illustrates another example tube support apparatus.

FIG. 19 illustrates an end portion and receiver pad of the example tube support apparatus shown in FIG. 18.

FIG. 20 illustrates the example receiver pad of FIGS. 18 and 19.

FIG. 21 illustrates an example end portion.

FIG. 22 schematically illustrates a protrusion in the channel of the receiver pad shown in FIGS. 18-20.

FIG. 23 illustrates another example tube support apparatus.

FIG. 24 illustrates an example clamp of the example tube support apparatus of FIG. 23.

FIG. 25 illustrates another view of the example tube support apparatus of FIG. 23.

FIG. 26 illustrates a top view of the example tube support apparatus of FIG. 23.

FIG. 27 illustrates a cross sectional view of the example support and clamp of FIGS. 23-26.

FIG. 28 illustrates a cross sectional view of another example support and clamp.

FIG. 29 illustrates an example tube holder.

FIG. 30 illustrates tubes and a cheek pad that may be used with the example tube holder of FIG. 29.

FIG. 31 illustrates the example tube holder of FIG. 29.

FIG. 32 illustrates the example tube holder of FIGS. 29 and 31.

FIG. 33 illustrates the example clip of the tube holder of FIGS. 29 and 31-32.

FIG. 34 illustrates another view of the example clip of FIG. 33.

FIG. 35 illustrates another example tube support apparatus.

FIG. 36 illustrates an example cheek pad and end portion of the example tube support apparatus of FIG. 35.

FIG. 37 illustrates another view of the example cheek pad of FIG. 36.

FIG. 38 illustrates the example tube support apparatus of FIG. 35.

FIG. 39 illustrates another view of the example cheek pad and end portion of the example tube support apparatus of FIG. 35.

FIG. 40 illustrates another example tube holder.

FIG. 41 illustrates another example tube holder.

FIG. 42 illustrates another example tube holder.

FIG. 43 illustrates an adhesive strip wrapped around an example tube.

### DETAILED DESCRIPTION

This application is related to apparatuses and methods for supporting endotracheal, feeding, gastric suction, and/or other tubes. In some prior art apparatuses, tape is used to secure the tube, such as an endotracheal tube, to a brace. Applicant has identified certain disadvantages of using tape. When infants are intubated and premature, they are often subjected to humidity for 30 or more days, which can make the tape less effective at securing and possibly lead to the tube coming out of position. Another drawback of tape is that medical professionals often have to remove their gloves before taping, removing the tape, or adjusting the tape, adding time and difficulty to the procedure. Still another drawback of tape is that intubated infants often vomit, which also can make the tape less effective at securing if the vomit contacts the tape. Yet another drawback of these prior art devices is that they often require 2-3 medical professionals to perform adjustments.

The example clamps and associated apparatuses and methods disclosed herein overcome the Applicant-identified disadvantages with prior art apparatuses, including those that use tape. For example, clamps with polymeric and/or elastomeric materials for securing a tube to a brace are relatively unaffected by humidity and vomit. Further, clamps with polymeric and/or elastomeric materials are easy to quickly adjust. In some examples, the clamps may lack metal so that patients can undergo procedures such as magnetic resonance imaging while remaining intubated. In some examples, the braces, clamps, and/or supports disclosed herein may be plastic. In some examples, the braces, clamps, and/or supports disclosed herein may be made of medical grade polypropylene. Further, the examples disclosed herein allow a single medical professional to make an adjustment. Examples disclosed further eliminate the need for paper or plastic tape measures to be placed against the lip of the patient for measuring and adjusting tube depth.

Further, the examples disclosed herein allow multiple tubes to be secured with one clamp at once, and further allow one of those tubes to be selectively removed or adjusted while the other tube remains secured, i.e., held in place longitudinally to control the depth of the tube in the patient. The benefits described herein are not limited to intubation procedures and may be achieved by other procedures, including feeding or gastric suction tube procedures. Further, while procedures regarding infants are described in some of the example embodiments, procedures for other patients of various ages may benefit from this disclosure.

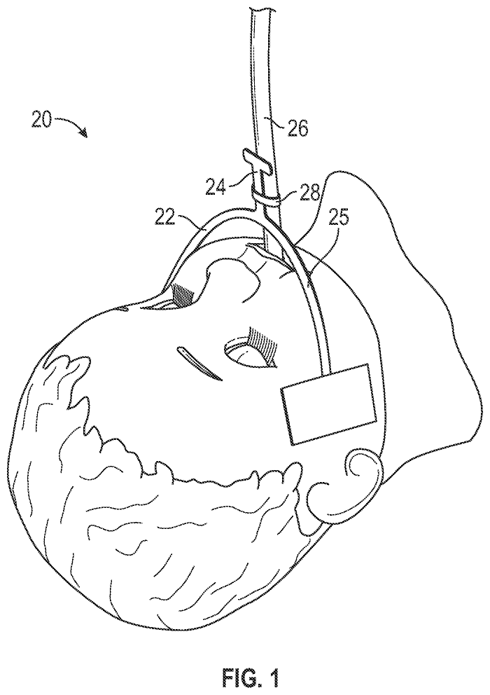

FIG. 1 illustrates an example tube support apparatus 20 including a brace 22 for attachment to a patient's face. The brace 22 may include a support 24 extending away from the patient's face as shown. The example support 24 extends from a curved portion 25 that is contoured to complement the patient's face and secured to the patient's face at its ends. A tube 26, such as an endotracheal tube, is received against the support 24 and placed through the mouth of the patient. A clamp 28 is provided to secure the tube 26 to the support 24 and surrounds both the tube 26 and the support 24. The example clamp 28 is made of one or more polymeric materials. The term polymer is used herein to refer generally to plastics, elastomers, thermoplastic elastomers, or other natural or synthetic materials that contain repeating molecule subunits. In some examples, the clamp 28 is plastic.

In the example shown, the patient is a newborn child, but intubations of other patients of various ages may benefit from this disclosure. Because of the small size of newborn children, the slightest adjustment in position of the tube can mean the difference of the infant receiving oxygen and not receiving oxygen. Accordingly, the examples disclosed herein provide improved securement of the endotracheal tube. Additionally or alternatively, procedures involving nasogastric tubes, orogastric tubes, and gastric suction may benefit from this disclosure.

In some prior art apparatuses, tape is used to secure the endotracheal tube to the brace. Applicant has identified certain disadvantages of using tape. When infants are intubated, they are often subjected to humidity, which can make the tape less effective at securing and possibly lead to the tube coming out of position. Another drawback of tape is that medical professionals often have to remove their gloves before taping, removing the tape, or adjusting the tape, adding time and difficulty to the procedure. Still another drawback of tape is that intubated infants often vomit, which also can make the tape less effective at securing if the vomit contacts the tape.

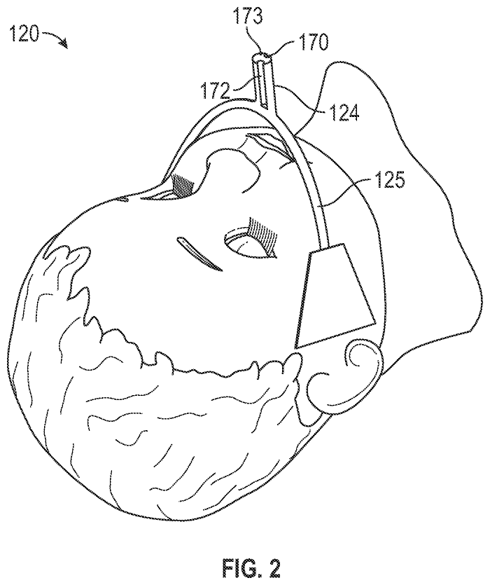

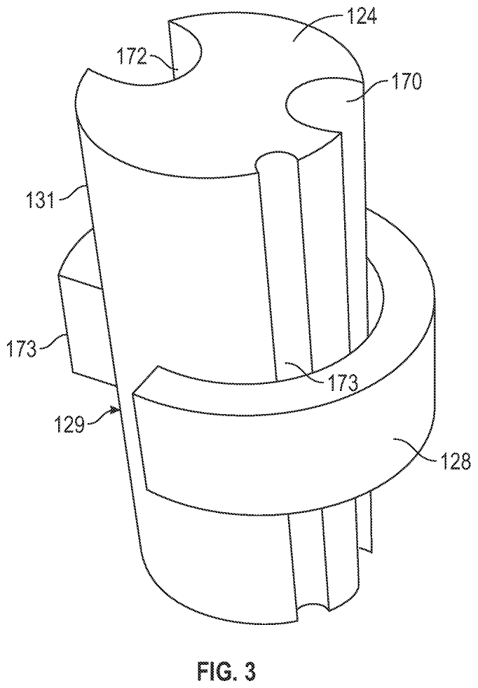

FIG. 2 illustrates another example tube support apparatus 120 including a support 124 providing grooves 170, 172, 173, for receiving tubes (removed for illustration purposes), including feeding and/or gastric suction tubes of different diameters, such as an orogastric tube and a nasogastric tube in some examples, as well as an endotracheal tube. In some examples, the tube support apparatus 120, including the example clamp 128 (see FIG. 3), is made of one or more polymeric materials. It should be understood that like reference numerals identify corresponding or similar elements throughout the several drawings. In some examples, the tube support apparatus 120, including the example clamp 128 (see FIG. 3), is made of plastic. In some examples, the tube support apparatus 120, including the example clamp 128 (see FIG. 3), is made of medical grade polypropylene.

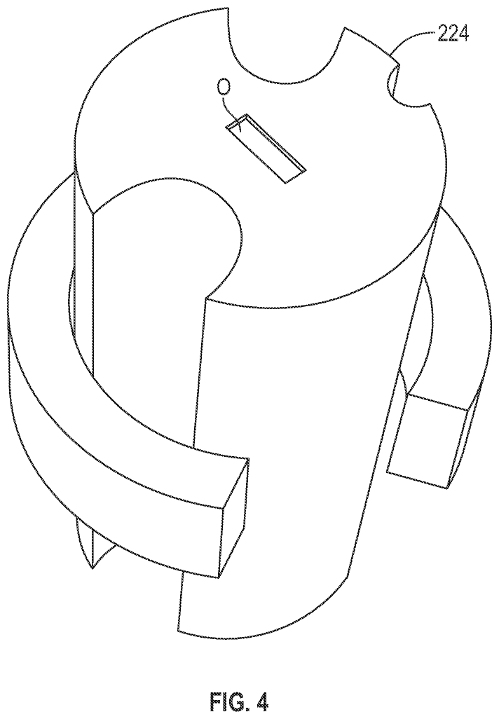

FIG. 3 illustrates a portion of the example support apparatus 120 of FIG. 2 including the support 124 and a clamp 128. In some examples, the support 124 may replace a support in an existing support apparatus, such as the support 24 in the embodiment in FIG. 1 or the equivalent support of the NeoTech® NeoBar®. In some examples, as shown in the embodiment in FIG. 4, a support 224 may be placed over such an existing support, such as by providing an opening O at the radial center of the support 224 for receiving the existing support.

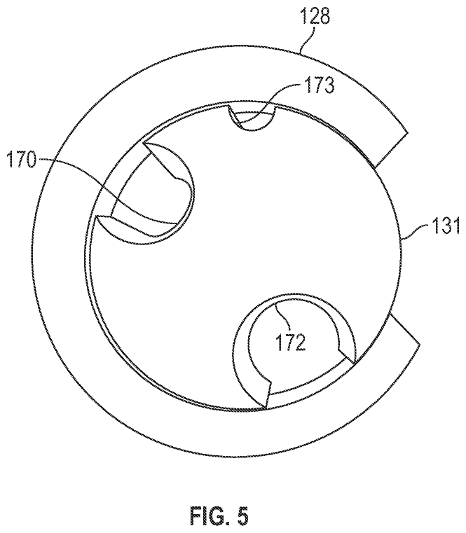

Referring back to FIG. 3, the clamp 128 is generally C-shaped, including an open circumferential portion 129. The open circumferential portion 129 is sized such that it is received over a radially outer surface 131 of the support 124 when tubes are to be secured in the grooves, such as by moving the clamp 128 in a direction generally perpendicular to the long axis of the support 124. When one tube is to be adjusted, replaced, etc., the clamp 928 may be rotated in a clockwise or counterclockwise direction so that the open portion 929 is circumferentially aligned with the groove in which that tube is received. Therefore, one tube can be adjusted, replaced, etc., while one or more other tubes remain secured in their respective grooves. In the example, the open portion 129 has an arc length greater than or equal to the arc length of the largest groove. The open portion 129 may also have an arc length that is less than or equal to the arc length of at least one of the radially outer surfaces of the support 124, such that the clamp 128 can be moved to a position in which all grooves are covered, such as that shown in FIG. 5.

FIG. 5 illustrates an engaged position of the example clamp 128 and support 124 in which tubes can be secured in all three grooves 170, 172, 173.

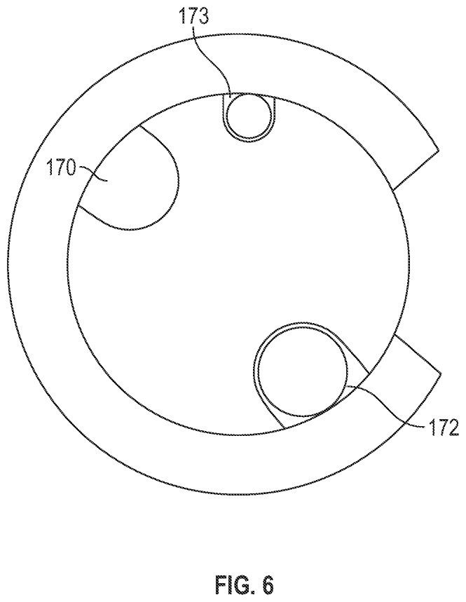

FIG. 6 illustrates the engaged position of the example clamp 128 and support 124 in which tubes are secured in grooves 170 and 172. As shown, the tubes can be compressed slightly and/or the clamp 128 can be expanded slightly in order to hold the tubes in place longitudinally to control the depth of the tube in the patient.

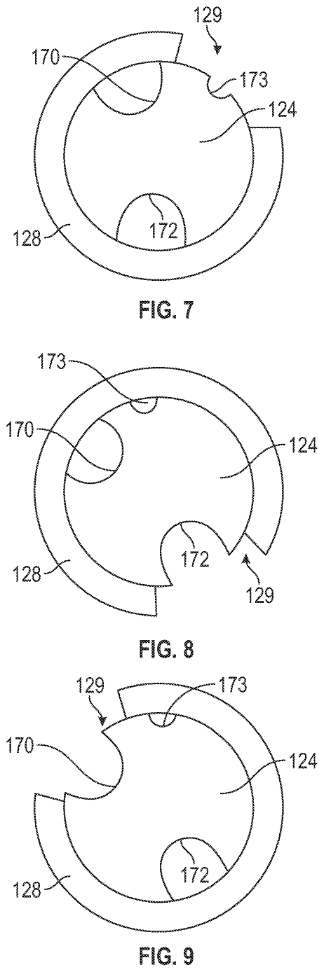

FIG. 7 illustrates a first disengaged position of the example clamp 128 and support 124, in which the open portion 129 is circumferentially aligned with the groove 173 and therefore the clamp 128 is disengaged with respect to the groove 173 such that a tube in the groove 173 can be removed, adjusted, etc., while tubes (not shown) in one or both of the grooves 172 and 170 remain secured.

FIG. 8 illustrates a second disengaged position of the example clamp 128 and support 124, in which the open portion 129 is circumferentially aligned with the groove 172 and therefore the clamp 128 is disengaged with respect to the groove 172 such that a tube in the groove 172 can be removed, adjusted, etc., while tubes in one or both of the grooves 170 and 173 remain secured.

FIG. 9 illustrates a third disengaged position of the example clamp 128 and support 124, in which the open portion 129 is circumferentially aligned with the groove 170 and therefore the clamp 128 is disengaged with respect to the groove 170 such that a tube in the groove 170 can be removed, adjusted, etc., while tubes in one or both of the grooves 173 and 172 remain secured.

While three grooves 170, 172, and 173 are shown in the examples, more or fewer grooves may be utilized in some examples. In some examples, other clamps could be used with the support 124. In some examples, as shown, the grooves 170, 172, and 173 are of different sizes to accommodate differently sized tubes.

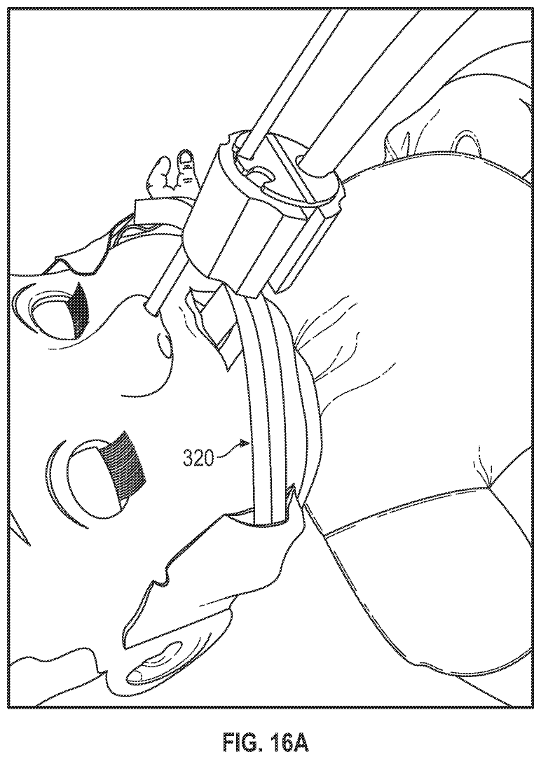

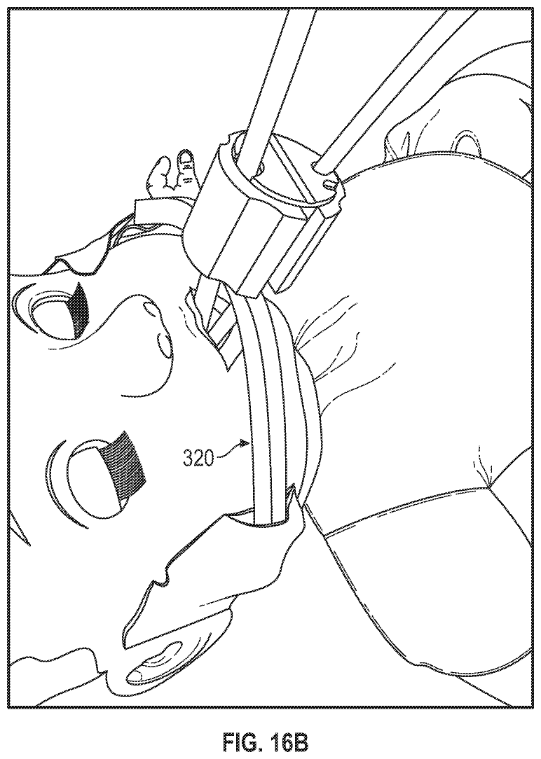

Referring back to FIG. 2, in the example shown, the apparatus 120 including curved portion 125 lies substantially in a plane, and grooves 170, 173 face one side of the plane, and the groove 172 faces an opposite side of the plane (see also FIG. 5). In the orientation shown, one of grooves 170, 173 can receive an orogastric tube, and the groove 172 receives an endotracheal tube. However, in an example in which a nasogastric tube is supported in one of the grooves 170, 173 instead of an orogastric tube, the orientation of the apparatus 120 can be flipped about 180° such that the grooves 170, 173 face the side of the plane that is nearer the nose. The example apparatus 320 discussed below has similar capability, and is further illustrated at FIGS. 16A and 16B. In some examples, as shown, the groove 173 is closer circumferentially to the groove 170 than to the groove 172.

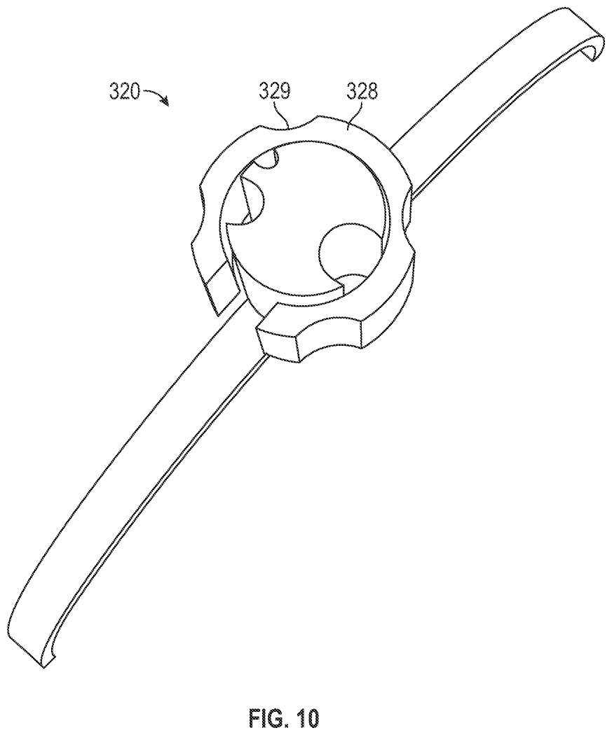

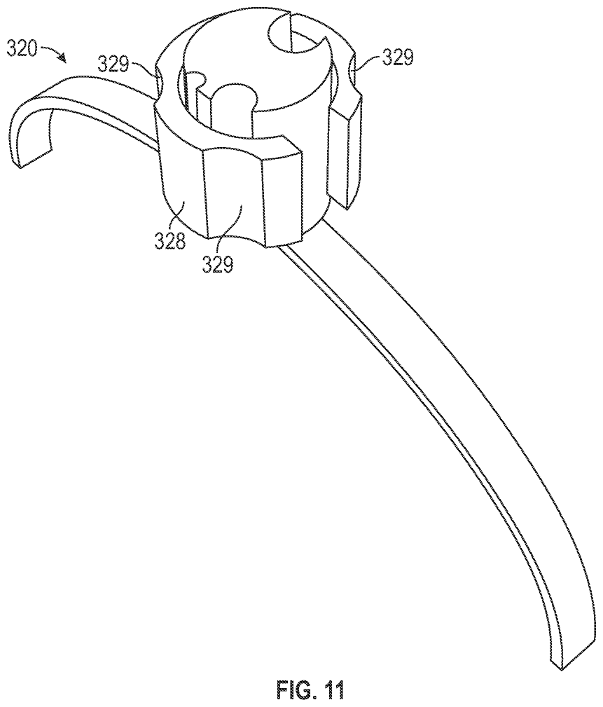

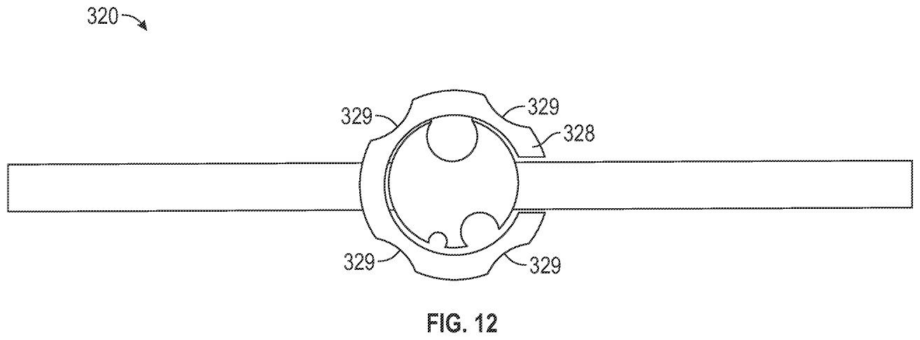

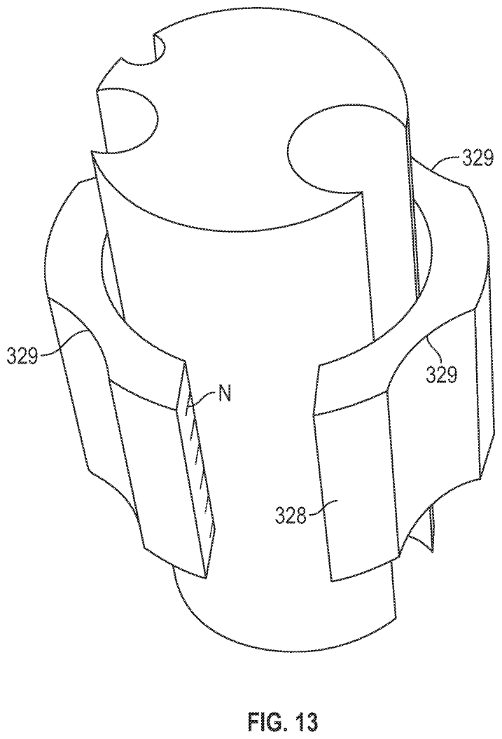

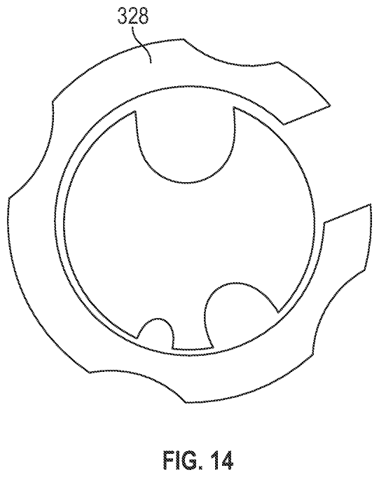

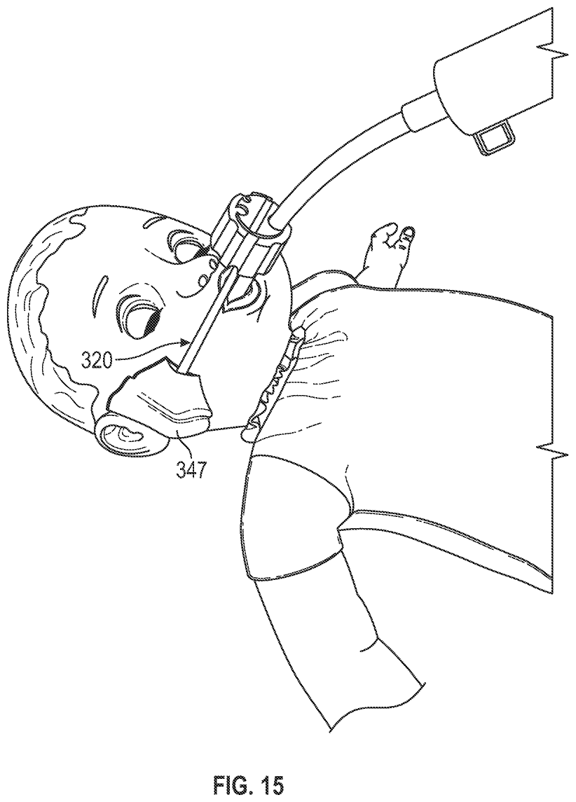

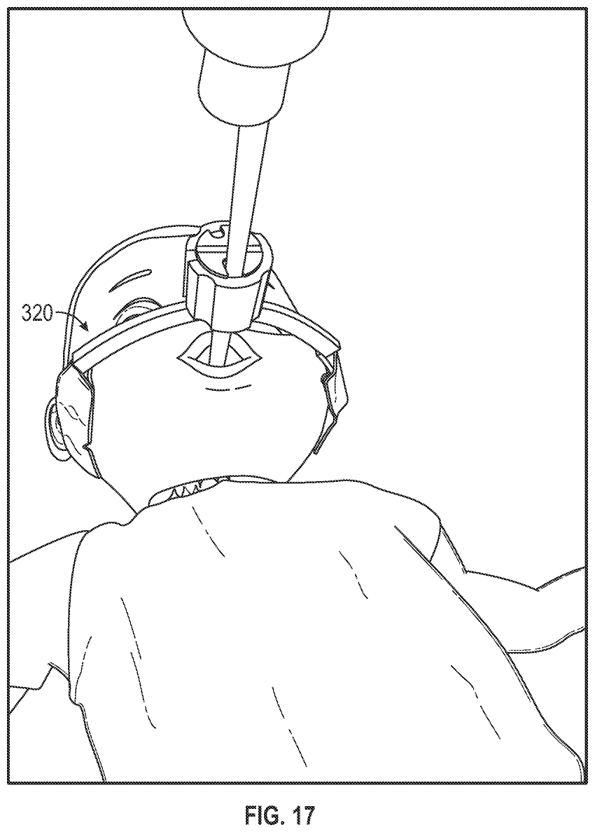

FIGS. 10-17 illustrate another example tube support apparatus 320, substantially similar to the tube support apparatus 120, except that the clamp 328 has outer grooves 329 on its radially outer surface for ease of grip, such as by medical professionals. FIGS. 15-17 illustrate the example tube support apparatus 320 positioned on a patient during intubation. In some examples, as shown, the pads 347 may be tapered. FIG. 16A illustrates an orientation of the example apparatus 320 in which a nasogastric tube is supported. FIG. 16B illustrates an orientation of the example apparatus 320 flipped 180 degrees from the orientation shown in FIG. 16A in which an orogastric tube is supported.

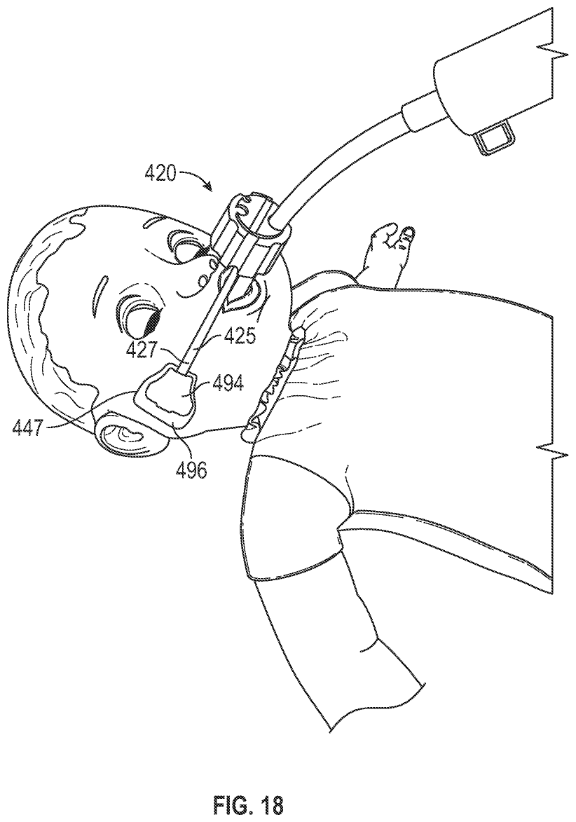

FIG. 18 illustrates another example tube support apparatus 420 in which the ends portions 427 of the curved portion 425 are received in a pad assembly 447, and more specifically in a receiver pad 494 adhered to an adhesive pad 496 adhered to the patient's skin proximal to the ear. In some examples, such as in infants, this area offers the most surface area for adhesion. In some examples, the pad assembly 447 is adhered in a position closer to the patient's ear than to the patient's mouth. Although one end of the curved portion is shown in one pad assembly 447 in the Figure, a similar arrangement is utilized proximal to the patient's other ear. In some examples, as shown, both the receiver pad 494 and the adhesive pad 496 are tapered, narrowing as they extend in the direction from the patient's ear to the mouth (medially), such as like the tapered pads previously discussed in this disclosure. In some examples, the receiver pad 494 includes silicone. In some examples, the adhesive pad 496 is a silicone tape adhesive pad. In some examples, the adhesive pad 496 is an acrylic adhesive pad. The example adhesive pad 496 may be larger than the example receiver pad 494 in some examples.

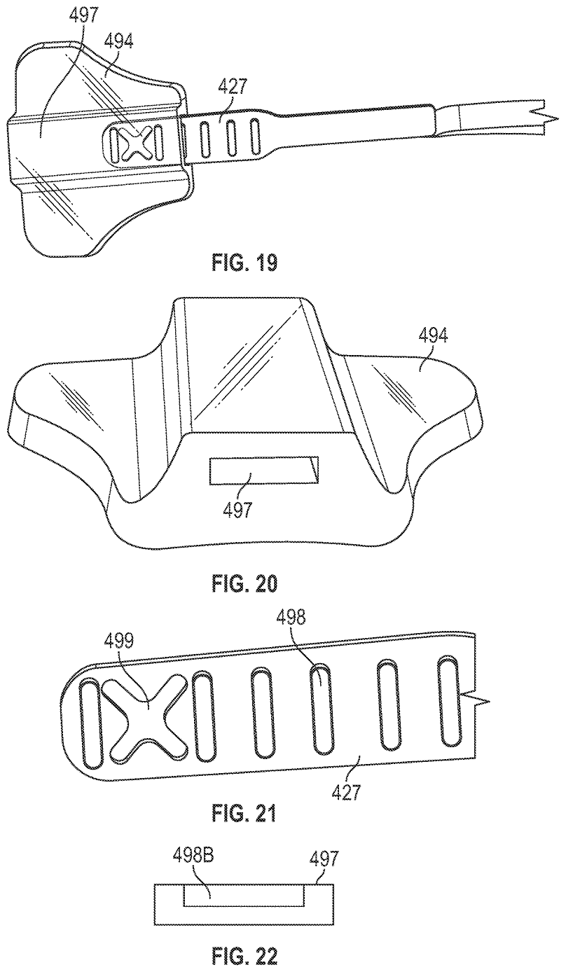

As shown in FIGS. 19 and 20, the end portion 427 is received in a channel 497 of the receiver pad 494. The end portion 427 is adjustable within the channel 497 to allow the example support apparatus 420 to be placed in an optimal position.

As shown in FIG. 21, the end portion 427 may include protrusions 498, such as ribs in some examples, for securement of the end portion 427 within the channel 497 (FIGS. 19 and 20). In some examples, as shown schematically in FIG. 22 the channel 497 may include one or more similar protrusions 498B, such as on its upper surface in some examples, for engaging with protrusions 498 on the end portion 427 and securing the end portion 427 in position once an optimal position is attained. An indicator 499 may be included on the end portion 427 to signal to a medical professional when to stop pulling the end portion 427 out of the channel 497 during an adjustment process to prevent the medical professional from pulling the end portion 427 completely out of the channel 497.

Regarding the example support apparatus 420 or other apparatus that use the pads 447, in an emergency, the brace can simply be pulled up and out of the pads 447. The pads 447 can remain on the patient's face until the emergency has been resolved. The brace can then be replaced into the pads 447 and reused. This is an improvement over some prior art devices which suggest the device be cut with scissors and pulled off the patient's face. Further, the example pads 447 are able to be cleaned with baby wipe or alcohol pad. Prior art pads could become soiled with vomit and/or saliva because they are cotton-topped and the whole apparatus would have to be changed out.

With reference to FIGS. 18-22, an example method may include one or more of the following steps: intubating a patient, placing a pad assembly 447 on the patient's cheek proximal to the patient's ear, placing a second pad assembly 447 on the patient's cheek proximal to the patient's other ear, positioning the end portions 427 of the curved portion 425 within the channels 497 of each pad assembly 447, adjusting the end portions 427 within the channels 497 of each pad assembly 447 to an optimal brace position, and securing the endotracheal tube within the example support apparatus 1120. Although an example support apparatus 420 is shown, other apparatuses, including other examples in this disclosure, may be utilized with the pad assembly 447. In some examples, there is no need for an additional adhesive to be placed over the top of the receiver pad 1194. Fewer or additional steps than are recited could be performed within the scope of this disclosure, and the recited order of steps is not intended to limit this disclosure.

In some examples, the grooves receiving the tubes may have indicators, such as notches or raised portions in some examples, for reference against the tubes for adjustment and positioning of the tubes. In some examples, the indicators may be spaced apart in equal intervals. In some examples, the intervals may be 0.25 cm. In some examples, as shown in FIG. 13, one or both of the circumferentially outer surfaces of the clamps 328/428 may have notches N spaced apart in the same or different intervals for adjustment precision.

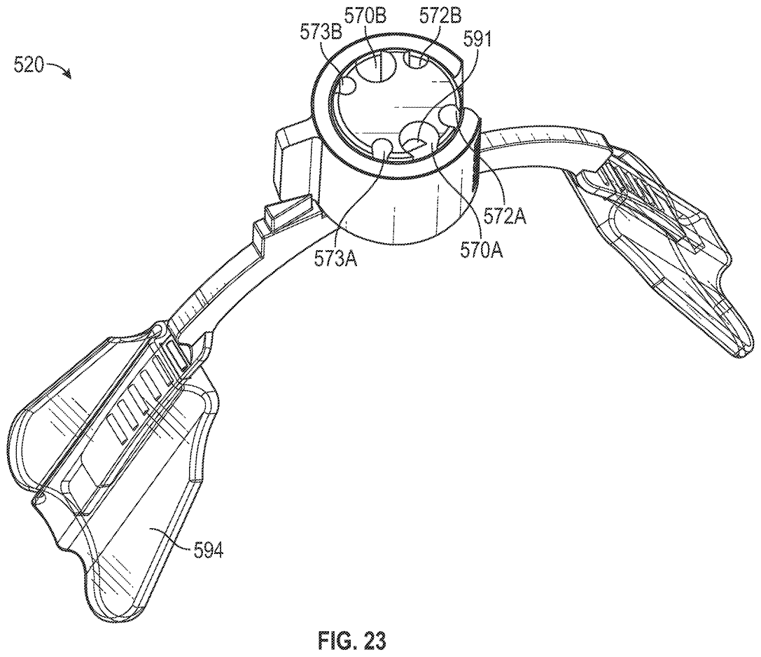

FIG. 23 illustrates another example support apparatus 520, substantially similar to the apparatuses 20/120/320/420, and including one or more protrusions for securing tubes within the grooves. Specifically, as shown in the example of FIG. 23, one or more protrusions 591 may extend radially outward (with respect to the support) from one or more of the grooves 570A/570B/572A/572B/573A/573B to be received against a tube (not shown) in the one or more grooves for securing the tube. Although six grooves are shown in the example, more or fewer grooves may be utilized. The example protrusion 591 is shelf-shaped, but other contours may be utilized. The example protrusion includes a straight edge extending from one circumferential position of the groove to a second circumferential position of the groove. Although one protrusion 591 in the groove 570A is shown, multiple axially-spaced protrusions within a groove may be utilized in some examples. Although the example protrusion 591 is shown in groove 570A in FIG. 23, any number of the grooves 570B/572A/572B/573A/573B may alternatively or additionally include one or more protrusions. The cheek pad 594 is the same as the cheek pad 494 in some examples. The example cheek pads 594 may be translucent and are shown as translucent for ease of viewing.

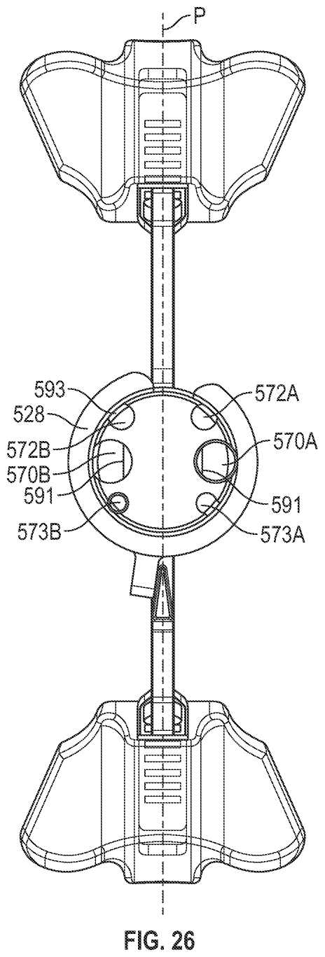

In some examples, as shown, the apparatus 520, including curved portion 525, lies substantially in a plane P (see FIG. 26), and grooves 570A, 572A, and 573A face one side of the plane, and the grooves 570B, 572B, 573B faces an opposite side of the plane. In some examples, when the apparatus is in place on a patient, the 570B, 572B, 573B may be open toward the superior direction, and the grooves 570A, 572A, and 573A may be open toward the inferior direction. Such an orientation may allow any tube to be inserted into the patient nasally or orally, or a combination of the two.

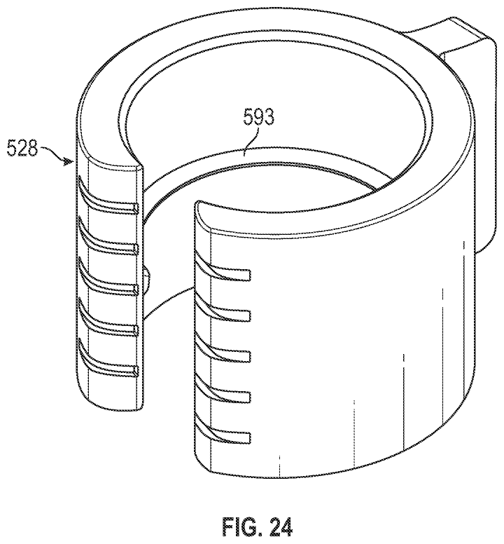

As shown in FIG. 24, alternatively or additionally, one or more protrusions 593 may extend radially inward from a radially inner surface of the clamp 528 to be received against one or more tubes (not shown) in the grooves for securing the tubes. In the example shown, the protrusion 593 is shelf-shaped and extends circumferentially along the radially inner surface of the clamp 528 and extends radially inward from the radially inner surface of the clamp 528. The example protrusion 593 is a C-shaped shelf with a curved radially inner edge. Other contours for the one or more protrusions 593 may be utilized in some examples.

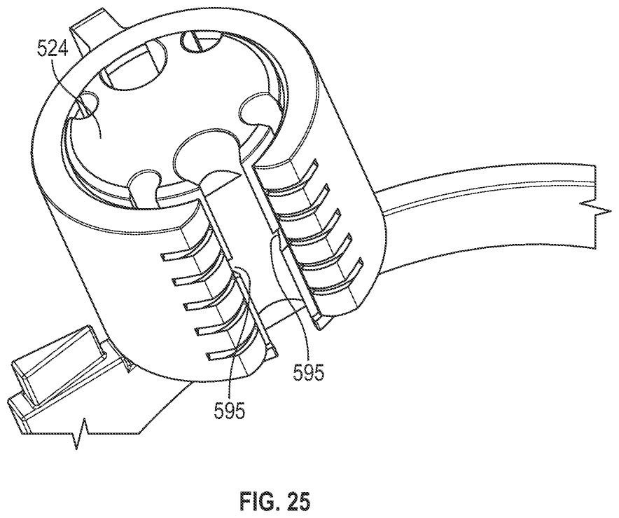

As shown in FIG. 25, the support 524 may include a circumferentially extending slot 595 extending radially inward from the radially outer surface of the support 524 to accommodate the protrusion 593, allowing the clamp 528 to be rotated about the support 524 with the protrusion 593 within the slot 595 and extending radially inward into the grooves 570A/570B/572A/572B/573A/573B. The example slot 595 extends about the circumference of the outer surface of the support 524 and into the grooves 570A/570B/572A/572B/573A/573B. In some examples, as shown, the protrusions 591 and 593 may be axially spaced from one another.

As shown in FIG. 26, the clamp 528 can therefore freely rotate relative to the support 524 with the protrusion 593 extending into the grooves 570A/570B/572A/572B/573A/573B for securing tubes within the grooves. The example protrusions 591 and 593 limit longitudinal movement of tubes within the grooves when in a secured position.

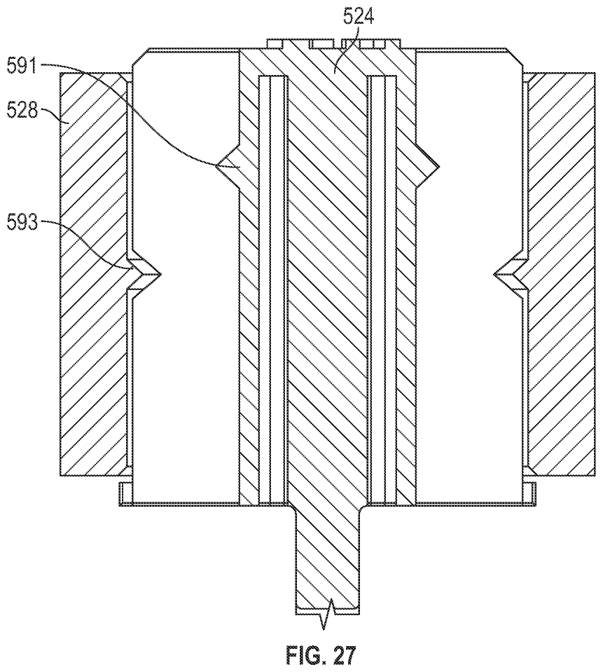

FIG. 27 illustrates a cross sectional view of an example support 524 and clamp 528. The body of the support 524 may be made of a first material and the protrusion 591 may be made of a second, different material. In some examples, the body of the support 524 is made of a higher durometer material than the protrusion 591. In some examples, the protrusion 591 is elastomeric. In some examples, the protrusion 591 is rubber. A low durometer (soft/gripping) material may secure the tubes from moving while also allowing for a larger range of tube sizes to be used. The support 524 may be made from a two shot molding process in some examples.

Similarly, the body of the clamp 528 may be made of a first material and the protrusion 593 may be made of a second, different material. In some examples, the body of the clamp 528 is made of a higher durometer material than the protrusion 593. In some examples, the protrusion 593 is elastomeric. In some examples, the protrusion 593 is rubber. A low durometer (soft/gripping) material may secure the tubes from moving while also allowing for a larger range of tube sizes to be used. The clamp 528 may be made from a two shot molding process in some examples.

As shown, the protrusion 591 may be axially spaced from the protrusion 593, which may result in better securement of tubes in some examples.

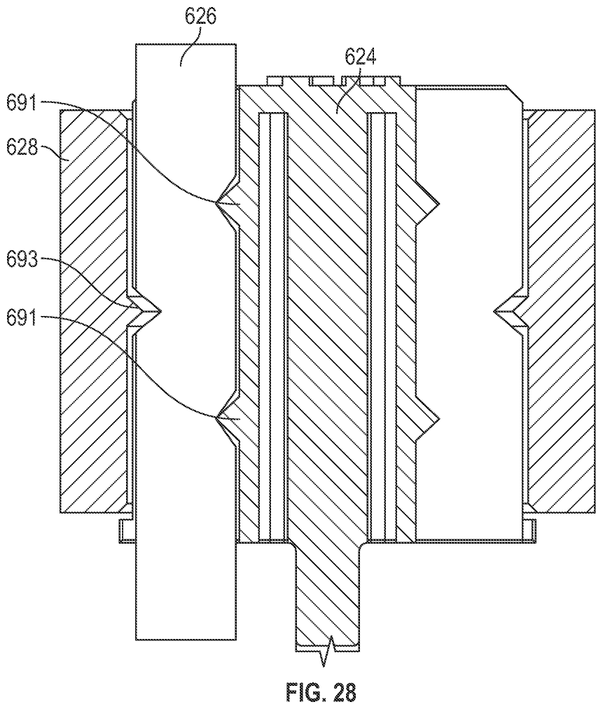

FIG. 28 schematically illustrates a tube 626 within another example support 624 and clamp 628. The support 624 includes two axially spaced protrusions 691. The protrusion 693 is axially between the protrusions 691, which Applicant has found results in improved securement of the tube in some applications, while also allowing for a larger range of tube sizes to be used.

An example tube support apparatus may be said to include a brace for attachment to a patient's face, a support extending from the brace and including a groove for receiving a tube, a first protrusion within the groove for being received against the tube, a clamp for surrounding the support and the tube, and a second protrusion extending from the clamp for being received against the tube. Although the protrusions are disclosed in the context of the examples shown in FIGS. 23-28, other tube support devices may benefit from the disclosed features.

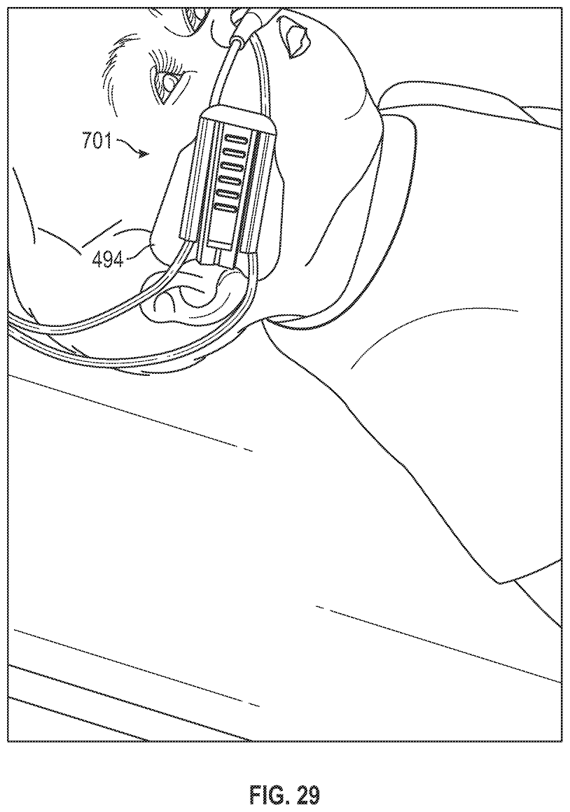

FIG. 29 illustrates a tube holder 701 utilizing one or both of the cheek pads 494. In some examples, the tube holder 701 is used for securing one or more of a nasal cannula, gastric suction tube, a feeding tube, and a respiratory support tube. In some examples, as the patient progresses from no longer being intubated, the patient may next have a nasal cannula, gastric suction, feeding tube, and/or any respiratory support tube and the same pad 494 can be used with the tube holder 701 as was used with the any of the tube support apparatuses disclosed herein, such that removal and reattachment of new pads is not warranted, reducing harm to the patient's skin. That is, a tube support apparatus, such as any of the examples disclosed herein, may be removed from the cheek pad, and the tube holder 701 may then be secured to the same cheek pad.

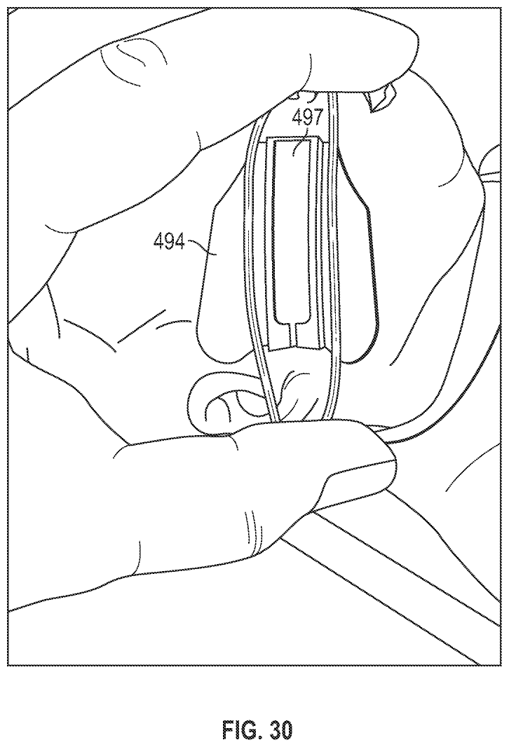

As shown in FIG. 30, when two tubes are held, the tubes may be placed on opposing sides of the raised surface providing the channel 497 (see FIG. 20). In other examples, such as when one tube is utilized, the tube may be placed on either side of the raised surface. The example pad 497 is shown as translucent for ease of viewing.

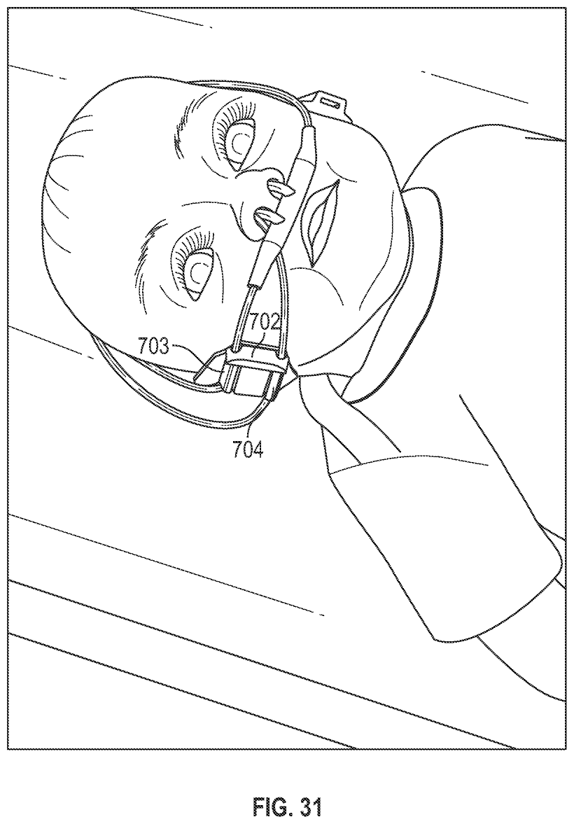

As shown in FIG. 31, an M-shaped clip 702 may be placed over the tubes and into the channel 497 to secure the tubes. The clip 702 includes outer legs 703, 704 for placement over the tubes.

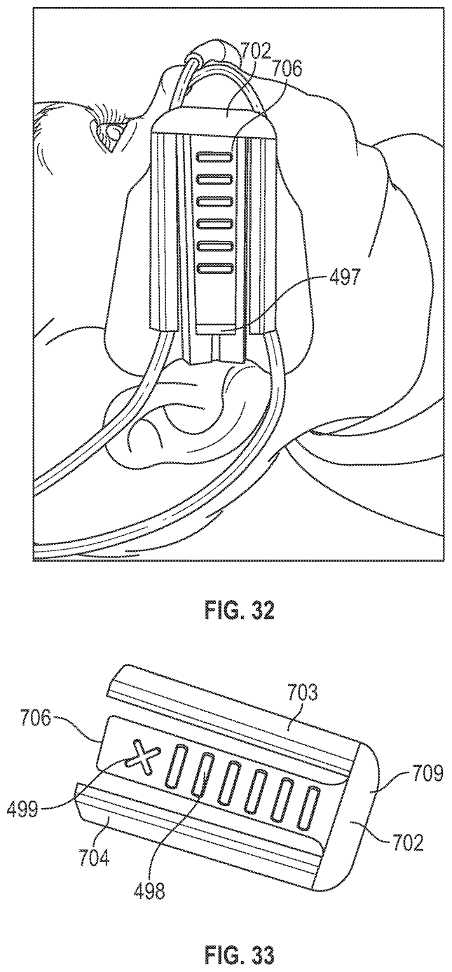

As shown in FIG. 32, the clip 702 includes a central leg 706 received in the channel 497. The leg 706 may be inserted into the channel 497 through a medial end of the channel 497 and moved in the lateral direction for securement. An end portion of a tube support apparatus (not shown) may be removed from the channel 497 in the medial direction before the leg 706 is inserted. The example pad 497 is shown as translucent for ease of viewing. The central leg 706 may have one or more of the same features of the end portions 427 (see FIG. 21), such as the protrusions 498 and indicator 499, as shown. The central leg 706 may have similar dimensions to the end portions 427. The central leg 706 may be secured within the channel 497 in a similar fashion to how the end portions 427 (not shown) are secured within the channel 497. The central leg 706 may provide the male engagement portion, and the channel 497 may provide the female engagement portion.

FIG. 33 illustrates the example clip 702 including the central leg 706 having protrusions 498 and indicator 499. The legs 703, 704, 706 extend from, and may be integrally formed with, a connecting portion 709 transverse to the legs 703, 704, 706. The clip 702 may be monolithic.

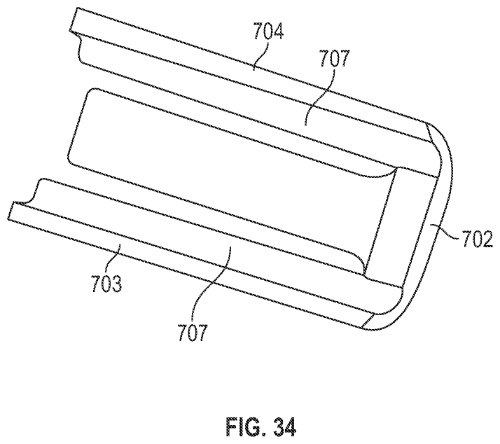

FIG. 34 illustrates the other side of the example clip 702 from that shown in FIG. 33. One or both of the legs 703, 704 include longitudinally extending grooves 707 for that complement the shapes of the tubes so that the grooves 707 may be received against the one or more tubes (not shown). The grooves 707 may extend longitudinally in the lateral direction.

In some examples, the clip 702 is polypropylene. In some examples, the clip 702 is silicone. In some examples, the clip 702 is made of a material that holds the tubes in place by friction.

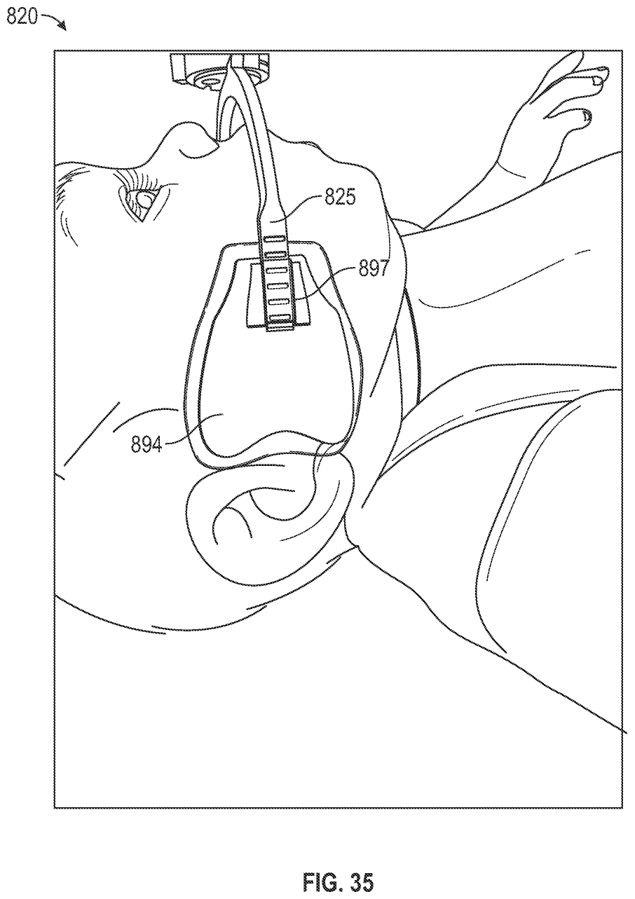

FIG. 35 illustrates another example support apparatus 820. In some examples, as shown, the channel 897 may not extend to the lateral end of the cheek pad 894. At least a portion of the end portion 825 may extend laterally beyond the channel 897 while secured.

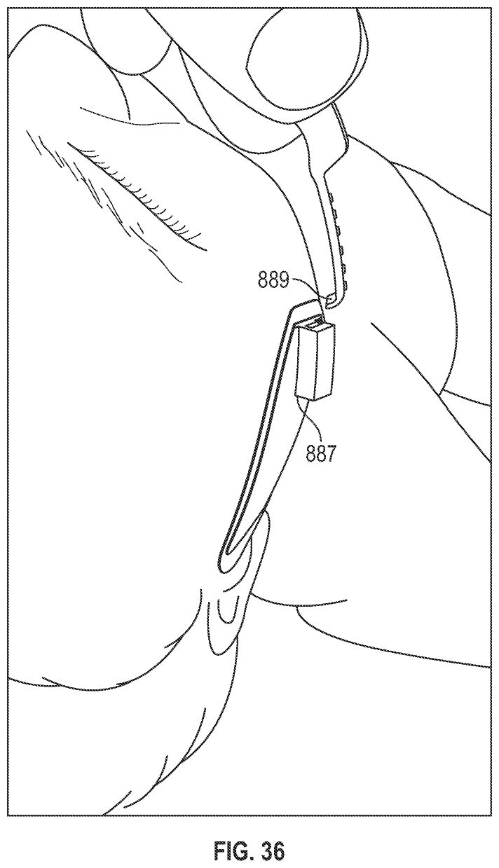

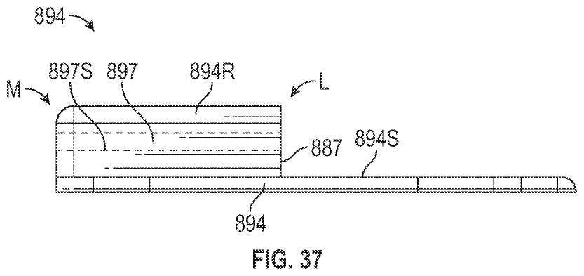

As shown in FIGS. 36 and 37, a raised edge 887 relative to the surrounding surface of the cheek pad 894 is therefore provided at the lateral end of the channel 897.

As shown in FIG. 37, the channel 897 may be a through opening extending from the medial end M to the lateral end L of the raised portion 894R of the cheek pad 894. The raised portion 894R is raised relative to a main body portion 894M. The lower surface 897S is raised relative to the upper surface 894S of the main body portion 894M of the cheek pad 894, providing the raised edge 887 at the lateral end L. The edge 887 may extend from the lateral end of the channel 897 to the surface 894S of the main body portion 894. Other edges, including edges at the lateral end of the raised portion 894R may be used to engage a support apparatus in other examples. The main body portion 894 may extend laterally farther than the raised portion 894R and channel 897, as shown.

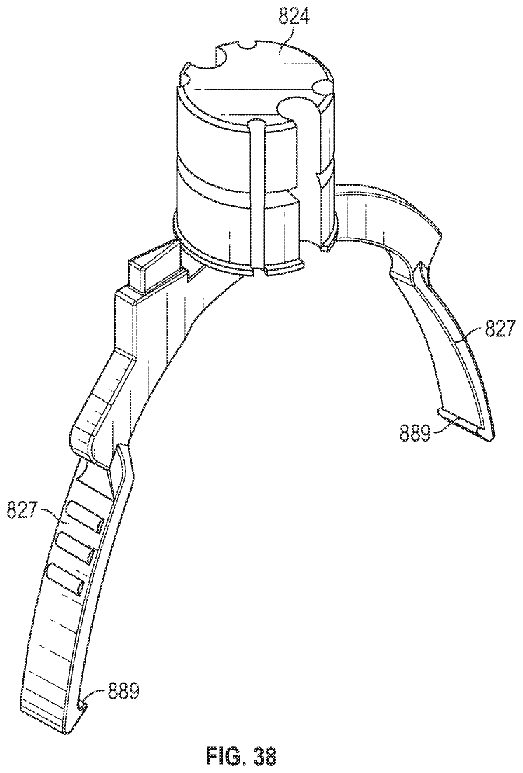

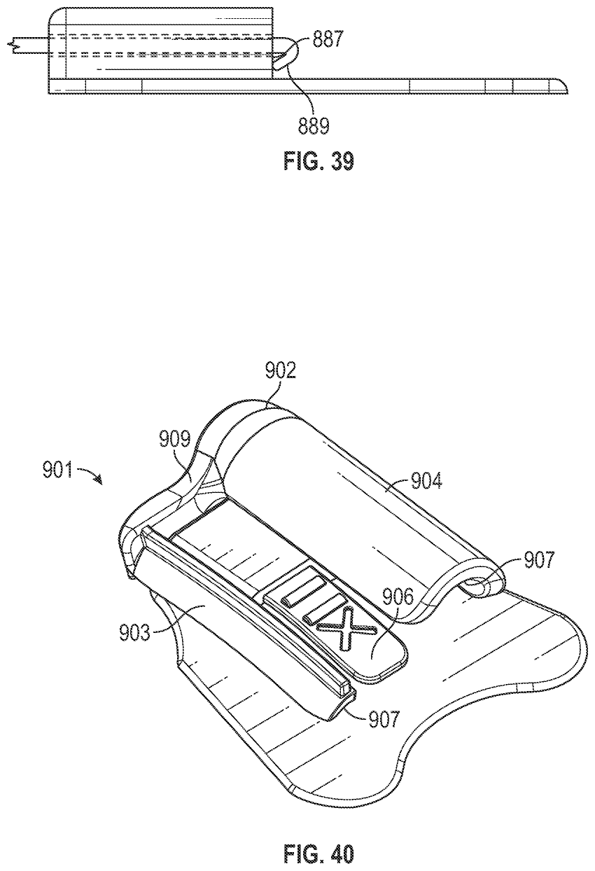

As shown in FIGS. 38 and 39, a protrusion 889 extends from one or both lateral ends of the end portion 827, so as to engage the raised edge 887 and inhibit the end portions 827 from being pulled out of their respective channels 897 under a certain threshold force. In some examples, as shown, the protrusion 889 may be angled to extend back toward the support 824 so as to hook the raised edge 887. In some examples, the protrusion 889 and raised edge 887 are configured such that the infant patient cannot pull the end portion 827 out of the channel 897 but a medical professional can if desired. In some examples, a protrusion may be configured to engage a different edge at the lateral end L of the channel than the one shown in the illustrative example.

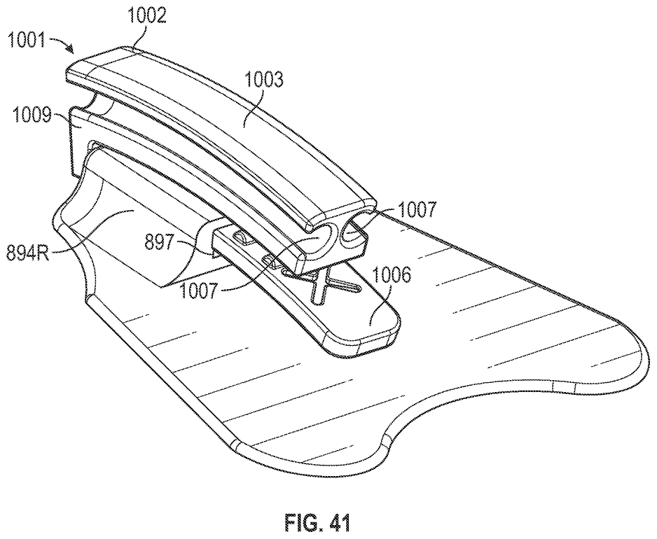

FIG. 40 illustrates another example clip 902 substantially similar to the example clip 702. The legs 903 and 904 and or their respective grooves 907 may be dimensioned differently, such as to accommodate different diameter tubes. The legs 903, 904, 906 extend from, and may be integrally formed with, a connecting portion 909. The clip 902 may be used with the cheek pads disclosed herein in some examples.

FIG. 41 illustrates another example clip 1002 substantially similar to the example clips 702 and 902. One leg 1003 provides two or more grooves 1007. The leg 1003 may be situated above the raised portion 894R when the leg 1006 is received within the channel 897. The grooves 1007 may be contoured and dimensioned such that tubes may be secured within the grooves 1007 by a press fit and without being secured against the pad. The clip 1002 may be used with the cheek pads disclosed herein in some examples.

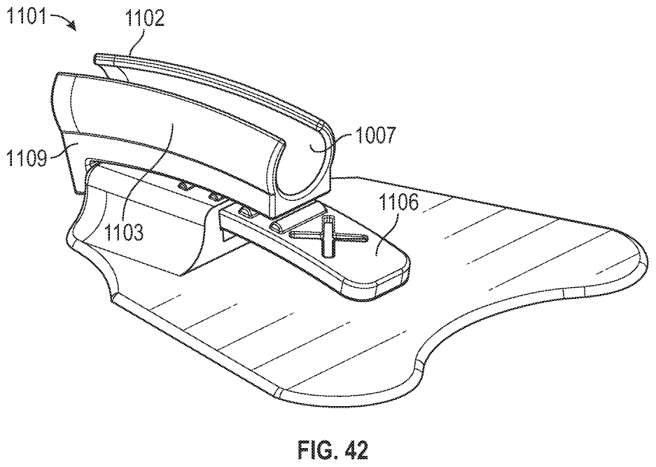

FIG. 42 illustrates another example clip 1102 substantially similar to the example clip 1002. The leg 1103 provides one groove 1107. The clip 1102 may be used with the cheek pads disclosed herein in some examples.

An example method in accordance with the examples of this disclosure may include removing a tube support apparatus from one or more cheek pads, placing one or more tubes in a patient, and securing the tubes to one or both of the example clip 702/902/1002/1102 and one or more of the cheek pads using the example clip 702. Fewer or additional steps than are recited could be performed within the scope of this disclosure, and the recited order of steps is not intended to limit this disclosure.

An example method of securing one or more tubes inserted within a patient may include removing an end portion of a tube support apparatus from a channel in a cheek pad, securing the one or more tubes by inserting a leg portion of a clip 702/902/1002/1102 into the channel of the same cheek pad and placing the one or more tubes within one or more grooves of the clip 702/902/1002/1102. Fewer or additional steps than are recited could be performed within the scope of this disclosure, and the recited order of steps is not intended to limit this disclosure.

Advantages associated with the tube holder 701/901/1001/1101 may include the elimination of tape on the face, such as across the upper lip and cheeks. Tape and other prior art holders need to be replaced frequently, sometimes daily. Skin damage may be reduced or eliminated from patients pulling the tubes out and ripping off the tape. When acrylic adhesive is utilized, it is not affected by heat and humidity, so the cheek pads won't lift in those environments. The cheek pads disclosed herein may be silicone, and silicone is cleanable, so when the patient vomits (a common occurrence, especially in infants) the pads can be wiped clean instead of replacing tape. In some examples, the tubes lay flat across the cheek with a physical, flat holder on top to lessen the chance of infant pulling the tubes out partially or all the way. One system may be provided that that is interchangeable between two respiratory support apparatuses. A stable cannula holder prevents nasal cannula tubing from curling down and nasal prongs coming out of the patient's nose (prevents patient oxygen levels from dropping due to oxygen coming out of nose). The tube holder 701 has a clean and organized look for parents and caregivers. The tube holder 701 prevents a feeding tube from becoming dislodged or pulled out, which can prevent aspiration pneumonia from misplaced tube.

Applicant has found that silicone provides the optimal amount of friction to prevent the tubes from moving in their respective grooves, while still being flexible enough to be comfortable on the patient's face. The tube holders and tube support apparatuses disclosed herein have been found to increase patient safety and comfort. A patient can be placed side lying or prone and the pad, clip, and or tube support apparatus are soft enough to conform to the patient's face.

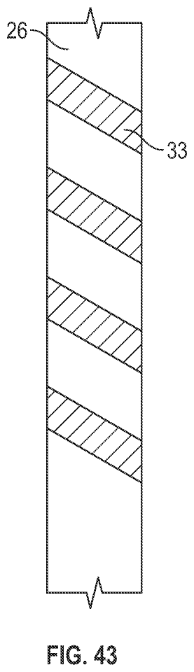

FIG. 43 illustrates adhesive 33 wrapped around a tube in a helical manner, which may be done in some examples to improve the interference fit between the tube and any of the devices disclosed herein.

In some examples, Applicant has found that Avery Dennison MED 5741 adhesive, or other similar adhesives, may be utilized to secure the cheek pads disclosed herein to the patient's skin.

Although the different examples are illustrated as having specific components, the examples of this disclosure are not limited to those particular combinations. It is possible to use some of the components or features from any of the embodiments in combination with features or components from any of the other embodiments.

The foregoing description shall be interpreted as illustrative and not in any limiting sense. A worker of ordinary skill in the art would understand that certain modifications could come within the scope of this disclosure. For these reasons, the following claims should be studied to determine the true scope and content of this disclosure.

What is claimed is:

1-20. (canceled)

21. A method of securing a tube support apparatus to a patient, the method comprising:
adhering a body lower surface of a cheek pad to a cheek of the patient, the cheek pad including:
a main body portion having a body upper surface and the body lower surface,
a raised portion extending from the body upper surface and defining a channel extending between a medial end and a lateral end of the raised portion, and
a lateral engagement surface at the lateral end of the raised portion; and
inserting an end portion of a brace into the channel from the medial end and through the lateral end of the channel, the end portion including a protrusion positioned to engage the lateral engagement surface to inhibit the end portion from being pulled out of the channel.

22. The method of claim 21, wherein the channel includes a channel lower surface that is raised relative to the body upper surface such that a raised edge extends from the lateral end of the channel to the body upper surface, and the lateral engagement surface comprises the raised edge, the protrusion being positioned to engage the raised edge to inhibit the end portion from being pulled out of the channel.

23. The method of claim 21, wherein the protrusion is angled toward a support of the tube support apparatus to hook the lateral engagement surface.

24. The method of claim 21, further comprising removing the end portion from the channel by applying a removal force to the brace to overcome engagement between the protrusion and the lateral engagement surface.

25. The method of claim 21, wherein the main body portion and the raised portion are formed of silicone.

26. The method of claim 25, wherein the body lower surface of the main body portion includes a medical adhesive.

27. The method of claim 21, wherein adhering the cheek pad includes adhering a first cheek pad to a first cheek of the patient and adhering a second cheek pad to an opposite cheek of the patient, the second cheek pad substantially similar to the first cheek pad, and the method further comprises:
inserting a second end portion of the brace into a channel of the second cheek pad such that a second protrusion on the second end portion engages a lateral engagement surface of the second cheek pad.

28. The method of claim 21, further comprising:
securing an endotracheal tube to a support of the tube support apparatus, the support extending from the brace.

29. The method of claim 28, further comprising:
securing at least one additional tube selected from a nasal cannula, a gastric suction tube, and a feeding tube to the support of the tube support apparatus.

30. A tube support assembly, comprising:
a brace for attachment to a face of a patient, the brace including an end portion having a protrusion; and
a cheek pad including:
a main body portion having a body upper surface and a body lower surface configured to be adhered to a cheek of the patient,
a raised portion extending from the body upper surface and defining a channel extending between a medial end and a lateral end of the raised portion, and
a lateral engagement surface at the lateral end of the raised portion;
wherein the end portion is received within the channel and the protrusion is positioned to engage the lateral engagement surface to inhibit the end portion from being pulled out of the channel.

31. The tube support assembly of claim 30, wherein the channel includes a channel lower surface that is raised relative to the body upper surface such that a raised edge extends from the lateral end of the channel to the body upper surface, and the lateral engagement surface comprises the raised edge.

32. The tube support assembly of claim 30, wherein the protrusion is angled to extend back toward a support of the brace so as to hook the lateral engagement surface.

33. The tube support assembly of claim 30, wherein the main body portion and the raised portion of the cheek pad are formed of silicone.

34. The tube support assembly of claim 33, wherein the body lower surface of the main body portion includes a medical adhesive.

35. The tube support assembly of claim 30, further comprising a support extending from the brace and including at least one groove for receiving a tube, and a clamp configured to secure the tube within the at least one groove.

36. A cheek pad for securing a tube support apparatus to a patient's face, the cheek pad comprising:
a main body portion having a body upper surface and a body lower surface configured to be adhered to a cheek of a patient; and
a raised portion extending from the body upper surface and defining a channel extending between a medial end and a lateral end of the raised portion, the raised portion further including a lateral engagement surface at the lateral end of the raised portion configured to be engaged by a protrusion on an end portion of a tube support apparatus to inhibit the end portion from being pulled out of the channel.

37. The cheek pad of claim 36, wherein the channel includes a channel lower surface that is raised relative to the body upper surface such that a raised edge extends from the lateral end of the channel to the body upper surface, and the lateral engagement surface comprises the raised edge.

38. The cheek pad of claim 36, wherein the channel is a through-opening extending from the medial end to the lateral end of the raised portion.

39. The cheek pad of claim 36, wherein the main body portion extends laterally beyond the raised portion.

40. The cheek pad of claim 36, wherein the main body portion and the raised portion are formed of silicone and the body lower surface includes a medical adhesive configured to adhere the cheek pad to the patient.
